(19) United States
(12) Patent Application Publication
(10) Pub. No.: US 2024/0229133 A1
(43) Pub. Date: Jul. 11, 2024

(54) METHODS AND COMPOSITIONS FOR SINGLE-STRANDED NUCLEIC ACID LADDERS BY GUIDED CLEAVAGE

(71) Applicant: Revvity Health Sciences, Inc., Waltham, MA (US)

(72) Inventors: Yanhong Tong, Boxford, MA (US); Zhixiang Lu, Waltham, MA (US)

(21) Appl. No.: 18/368,415

(22) Filed: Sep. 14, 2023

Related U.S. Application Data
(60) Provisional application No. 63/406,513, filed on Sep. 14, 2022.

(51) Int. Cl. C12Q 1/6876; C12P 19/34

(52) U.S. Cl. CPC C12Q 1/6876; C12P 19/34; C12Q 2600/166

(57) ABSTRACT

The present disclosure relates to methods, compositions, and kits for providing cleaved, single-stranded nucleic acids. Such nucleic acids can be used to provide ladder compositions.

### CROSS-REFERENCE TO RELATED APPLICATION

This application claims priority to U.S. Provisional Application Ser. No. 63/406,513, filed Sep. 14, 2022. The entire contents of which are hereby incorporated by reference.

### SEQUENCE LISTING

This application contains a Sequence Listing that has been submitted electronically as an XML file named 25947-0067001_SL_ST26.xml. The XML file, created on Sep. 15, 2023, is 4,644 bytes in size. The material in the XML file is hereby incorporated by reference in its entirety.

### FIELD

The present disclosure relates to methods, compositions, and kits for providing cleaved, single-stranded nucleic acids. Such nucleic acids can be used to provide ladder compositions.

### BACKGROUND

Single-stranded deoxyribonucleic acid (ssDNA) or ribonucleic acid (ssRNA) is used in a variety of biotechnology applications, including but not limited to genome editing, gene synthesis, gene therapy, and drug delivery. Detection and size analysis of single-stranded nucleic acid molecules can be accomplished by a variety of techniques, e.g., electrophoresis in agarose or polyacrylamide gels, capillary electrophoresis, microfluidic chip, and the like.

Nucleic acid-based molecular weight standards (or ladders) are useful tools for estimating the quality, size, and/or quantity of a nucleic acid sample. A standard is typically fractionated simultaneously with the sample (e.g., in parallel with the sample). Following detection, a comparison is made between the sample band(s) and the bands of the standard. Knowing the size (e.g., in nucleotides or nt) or other characteristics of the standard allows the size of unknown fragment(s) to be estimated.

### SUMMARY

The present disclosure relates to methods, compositions, and kits including cleaved, single-stranded nucleic acids. In particular embodiments, such nucleic acids can be useful as ladders that contain single-stranded DNA (ssDNA), single-stranded RNA (ssRNA), or single-stranded DNA/RNA hybrids (ssDNA/RNA).

In particular embodiments, the methods herein allow for single-stranded nucleic acids (e.g., ssDNA, ssRNA, or ssDNA/RNA) to be prepared in quantities and conditions that provide ladder compositions. For instance, typical methods to prepare double-stranded DNA (dsDNA) are not amenable for preparing ssDNA ladders. In one example, digestion of known-length dsDNA can be used to create ladder fragments, in which the lengths of ladder fragments are dependent on restriction enzyme sites. In another example, polymerase chain reaction (PCR) can be used to generate dsDNA having defined lengths. Such processes are limited, e.g., by the length of fragments that can be generated, in which long fragments (e.g., more than 500 bases) are difficult to generate. Furthermore, such processes can require removal of certain components prior to use as a ladder. For instance, if an ssDNA ladder is required, then generated dsDNA fragments will need to be denatured or otherwise treated to provide the single-stranded form and/or to remove undesired complementary sequences. In another instance, PCR requires additional reagents (e.g., polymerase, salts, other excipients, etc.), which may be preferable to remove prior to use as a ladder. Thus, in some non-limiting embodiments, the methods herein allow for nucleic acids to be prepared with sufficient lengths (e.g., more than 500 bases) and/or desired conformation (e.g., single-stranded conformation).

The present disclosure also relates to methods for generating single-stranded nucleic acids with defined or predetermined size (e.g., length). In some embodiments, the methods herein can employ oligonucleotide-guided cleavage of single-stranded templates by cleavage enzymes (e.g., restriction enzyme, nicking enzyme, programmable endonuclease, Argonaute protein, Cas enzyme, as well as others described herein). The guided oligonucleotide (or guides) can be ssDNA, ssRNA, or ssDNA/RNA. Using such guides, a double-stranded structure can be formed between the guide and the template, and the cleavage enzyme can be recruited to bind to double-stranded structures. If a cut site (e.g., a restriction site, a recognition sequence, a protospacer region, a protospacer adjacent motif (PAM) sequence, or a reverse complement thereof) is present in the template, then the cleavage enzyme can be used to cleave such cut sites. In this manner, the method can provide a plurality of cleaved template molecules (e.g., a plurality of cleaved, single-stranded nucleic acids).

In some embodiments, the guided oligonucleotide has a sufficiently short length, such that separation of such guides is not required for using the cleaved template molecules as ladders. Non-limiting lengths for guided oligonucleotides include less than about 40, 30, 20, or 10 nucleotides (nt); or from about 10 to 40 nt (e.g., from about 10 to 38 nt, 10 to 36 nt, 10 to 34 nt, 10 to 32 nt, 10 to 30 nt, 10 to 29 nt, 10 to 28 nt, 10 to 27 nt, 10 to 26 nt, 10 to 25 nt, 10 to 24 nt, 10 to 23 nt, 10 to 22 nt, 10 to 21 nt, 10 to 20 nt, 12 to 40 nt, 12 to 38 nt, 12 to 36 nt, 12 to 34 nt, 12 to 32 nt, 12 to 30 nt, 12 to 29 nt, 12 to 28 nt, 12 to 27 nt, 12 to 26 nt, 12 to 25 nt, 12 to 24 nt, 12 to 23 nt, 12 to 22 nt, 12 to 21 nt, 12 to 20 nt, 14 to 40 nt, 14 to 38 nt, 14 to 36 nt, 14 to 34 nt, 14 to 32 nt, 14 to 30 nt, 14 to 29 nt, 14 to 28 nt, 14 to 27 nt, 14 to 26 nt, 14 to 25 nt, 14 to 24 nt, 14 to 23 nt, 14 to 22 nt, 14 to 21 nt, 14 to 20 nt, 16 to 40 nt, 16 to 38 nt, 16 to 36 nt, 16 to 34 nt, 16 to 32 nt, 16 to 30 nt, 16 to 29 nt, 16 to 28 nt, 16 to 27 nt, 16 to 26 nt, 16 to 25 nt, 16 to 24 nt, 16 to 23 nt, 16 to 22 nt, 16 to 21 nt, 16 to 20 nt, 18 to 40 nt, 18 to 38 nt, 18 to 36 nt, 18 to 34 nt, 18 to 32 nt, 18 to 30 nt, 18 to 29 nt, 18 to 28 nt, 18 to 27 nt, 18 to 26 nt, 18 to 25 nt, 18 to 24 nt, 18 to 23 nt, 18 to 22 nt, 18 to 21 nt, 18 to 20 nt, 20 to 40 nt, 20 to 38 nt, 20 to 36 nt, 20 to 34 nt, 10 to 32 nt, 20 to 30 nt, 20 to 29 nt, 20 to 28 nt, 20 to 27 nt, 20 to 26 nt, 20 to 25 nt, 20 to 24 nt, 20 to 23 nt, 20 to 22 nt, 20 to 21 nt, and the like).

The present disclosure also provides nucleic acid compositions (or ladders) that may be used as standards for estimating the size (e.g., in nt for a chain length of a single-stranded polynucleotide) and/or mass of nucleic acid molecules of unknown size and/or mass. Accordingly, the present disclosure relates to methods for producing such compositions or ladders. Additional details follow.

### Definitions

As used herein, the term “about” means+/−10% of any recited value. As used herein, this term modifies any recited value, range of values, or endpoints of one or more ranges.

As used herein, the term “cleavage” or “cleave” is meant the breakage of the covalent backbone in a template (e.g., a target region of the template). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Cleavage can result in the production of blunt ends, staggered ends (or sticky ends), or nicked ends. Furthermore, cleavage can occur in DNA, RNA, and DNA/RNA hybrid sequences or regions. In some but not all embodiments, cleavage can be associate with breakage of the covalent backbone in a guided oligonucleotide.

“Complementarity” or “complementary” or like terms refer to the ability of a nucleic acid to form hydrogen bond(s) with another nucleic acid sequence by either traditional Watson-Crick or other non-traditional types, e.g., form Watson-Crick base pairs and/or G/U base pairs, “anneal,” or “hybridize” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” or “sufficient complementarity” or “sufficiently complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Other features and advantages of the present disclosure will be apparent from the following detailed description, the figures, and the claims.

Stringent conditions for hybridization refer to conditions under which a nucleic acid having complementarity to a target region predominantly hybridizes with the target region, and substantially does not hybridize to non-target regions. Stringent conditions are generally sequence-dependent, and vary depending on a number of factors. In general, the longer the sequence, the higher the temperature at which the sequence specifically hybridizes to its target region. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

As used herein, the term “fragment” when referring to a nucleic acid is meant a portion of a nucleic acid that is at least one nucleotide shorter than the reference sequence. This portion contains, preferably, at least about 10%, 20%, 30%, 40%, 50%, 60%, 70%, 80%, or 90% of the entire length of the reference nucleic acid molecule. A fragment may contain 10, 20, 30, 40, 50, 60, 70, 80, 90, or 100, 200, 300, 400, 500, 600, 700, 800, 900, 1000, 1250, 1500, 1750, 1800 or more nucleotides. In another example, any nucleic acid fragment can include a stretch of at least about 5 (e.g., about 10, about 20, about 30, about 40, about 50, or about 100) nucleotides that are at least about 40% (e.g., about 50%, about 60%, about 70%, about 80%, about 90%, about 95%, about 87%, about 98%, about 99%, or about 100%) identical to any of the sequences described herein can be utilized in accordance with the disclosure. In yet another example, any nucleic acid fragment can include a stretch of at least about 5 (e.g., about 7, about 8, about 10, about 12, about 14, about 18, about 20, about 24, about 28, about 30, or more) nucleotides that are at least about 40% (about 50%, about 60%, about 70%, about 80%, about 90%, about 95%, about 87%, about 98%, about 99%, or about 100%) identical to any of the sequences described herein can be utilized in accordance with the disclosure.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as denaturation at a high temperature (e.g., more than 90° C.) and/or annealing with certain speeds (e.g., ramping speeds) of cool down. A sequence capable of hybridizing with a given sequence is referred to as the “complement” of the given sequence. Hybridization and washing conditions are well known and exemplified in Sambrook J, Fritsch E F, and Maniatis T, “Molecular Cloning: A Laboratory Manual,” Second Edition, Cold Spring Harbor Laboratory Press, Cold Spring Harbor (1989), particularly Chapter 11 and Table 11.1 therein; and Sambrook J and Russell W, “Molecular Cloning: A Laboratory Manual,” Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides), the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and hybridization solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul S F et al., J. Mol. Biol. 1990; 215:403-10; Zhang J et al., Genome Res. 1997; 7:649-56) or by using the Gap program (Wisconsin Sequence Analysis Package, Version 8 for Unix, Genetics Computer Group, University Research Park, Madison Wis.), using default settings, which uses the algorithm of Smith T F et al., Adv. Appl. Math. 1981; 2(4):482-9).

As used herein, “ladder” or “size standard” may be used interchangeably, and generally refers to a set of standards that are used to identify the approximate size and/or mass of a molecule. In certain embodiments, identification of size can include electrophoresis, using the principle that molecular weight is inversely proportional to migration rate through a gel matrix. In some embodiments, when used in electrophoresis, ladders can provide a linear scale or a logarithmic scale by which to estimate the size or mass of unknown fragments (providing the fragment sizes of the marker are known).

By “linker” is meant any useful multivalent (e.g., bivalent) component useful for joining to different portions or segments. Exemplary linkers include a nucleic acid sequence, a chemical linker, etc. In one instance, the linker can have a length of from about 3 nucleotides (nt) to 100 nt. For example, the linker can have a length of from about 3 nt to 90 nt, 3 nt to 80 nt, 3 nt to 70 nt, 3 nt to 60 nt, 3 nt to 50 nt, 3 nt to 40 nt, 3 nt to 30 nt, 3 nt to 20 nt, or 3 nt to 10 nt. In another example, the linker can have a length of 3 nt to 5 nt, 5 nt to 10 nt, 10 nt to 15 nt, 15 nt to 20 nt, 20 nt to 25 nt, 25 nt to 30 nt, 30 nt to 35 nt, 35 nt to 40 nt, 40 nt to 50 nt, 50 nt to 60 nt, 60 nt to 70 nt, 70 nt to 80 nt, 80 nt to 90 nt, or 90 nt to 100 nt.

The terms “nicking enzyme” or “nicking endonuclease” or “nickase” may be used interchangeably and generally refers to an enzyme that cuts one strand of a double-stranded DNA at specific recognition nucleotide sequences known as a restriction site. Such enzymes hydrolyze (cut) only one strand of the DNA duplex, thereby producing DNA molecules that are “nicked” or cleaved within only one of the strands (e.g., only the top strand or only the bottom strand).

The terms “nuclease” and “endonuclease” are used interchangeably herein to mean an enzyme that possesses catalytic activity for DNA cleavage and/or RNA cleavage. Examples of nucleases include restriction enzymes, nicking enzymes, programmable endonucleases, Argonaute proteins, Cas enzymes, or variants thereof. A variant can include a mutant of a wild-type version of a nuclease (e.g., substitutions at one or more amino acids, such as conservative amino acid substitutions), in which the catalytic activity for DNA cleavage and/or RNA cleavage is increased, decreased, or retained but in which this activity is not abolished. In general, described herein are various sites and motifs (e.g., double-stranded structures) useful for recruiting a nuclease to a complex including a guided oligonucleotide that is bound (or hybridized) to a template. Such sites and motifs can include one or more cut locations due to cleavage by a nuclease and/or can include one or more locations to bind a nuclease. Non-limiting examples of such recruiting sites or motifs includes a restriction site (e.g., for binding and/or cleavage by a restriction enzyme or a nicking enzyme), a recognition site (e.g., for binding and/or cleavage by a nuclease, such as a programmable endonuclease including an Argonaute protein), a protospacer region (e.g., for cleavage by a nuclease, such as a programmable endonuclease including a Cas enzyme), and/or a protospacer adjacent motif (PAM) sequence (e.g., for binding by a nuclease, such as a programmable endonuclease including a Cas enzyme).

The terms “polynucleotide” and “nucleic acid,” used interchangeably herein, refer to a polymeric form of nucleotides of any length, either ribonucleotides or deoxyribonucleotides. Thus, this term includes, but is not limited to, single-stranded (e.g., sense or antisense), double-stranded, or multi-stranded ribonucleic acids (RNAs), deoxyribonucleic acids (DNAs), threose nucleic acids (TNAs), glycol nucleic acids (GNAs), peptide nucleic acids (PNAs), locked nucleic acids (LNAs), or hybrids thereof, genomic DNA, cDNA, or DNA/RNA hybrids, as well as a polymer comprising purine and pyrimidine bases or other natural, chemically or biochemically modified, non-natural, or derivatized nucleotide bases. Polynucleotides can have any useful two-dimensional or three-dimensional structure or motif, such as regions including one or more duplex, triplex, quadruplex, hairpin, and/or pseudoknot structures or motifs. When N is used in a nucleic acid sequence, it can be represented by any nucleic acid (e.g., G, C, A, T, or U, as well as modified forms thereof).

The term “modified,” as used in reference to nucleic acids, means a nucleic acid sequence including one or more modifications to the nucleobase, nucleoside, nucleotide, phosphate group, sugar group, and/or internucleoside linkage (e.g., phosphodiester backbone, linking phosphate, or a phosphodiester linkage).

The nucleoside modification may include, but is not limited to, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

A sugar modification may include, but is not limited to, a locked nucleic acid (LNA, in which the 2′-hydroxyl is connected by a C₁₋₆ alkylene or C₁₋₆ heteroalkylene bridge to the 4′-carbon of the same ribose sugar), replacement of the oxygen in ribose (e.g., with S, Se, or alkylene, such as methylene or ethylene), addition of a double bond (e.g., to replace ribose with cyclopentenyl or cyclohexenyl), ring contraction of ribose (e.g., to form a 4-membered ring of cyclobutane or oxetane), ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone), multicyclic forms (e.g., tricyclic), and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with a-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

A backbone modification may include, but is not limited to, 2′-deoxy- or 2′-O-methyl modifications. A phosphate group modification may include, but is not limited to, phosphorothioate, phosphoroselenates, boranophosphates, boranophosphate esters, hydrogen phosphonates, phosphoramidates, phosphorodiamidates, alkyl or aryl phosphonates, phosphotriesters, phosphorodithioates, bridged phosphoramidates, bridged phosphorothioates, or bridged methylene-phosphonates.

The term “protein,” “peptide,” or “polypeptide,” as used interchangeably, is meant any chain of more than two amino acids, regardless of post-translational modification (e.g., glycosylation or phosphorylation), constituting all or part of a naturally occurring polypeptide or peptide, or constituting a non-naturally occurring polypeptide or peptide, which can include coded amino acids, non-coded amino acids, modified amino acids (e.g., chemically and/or biologically modified amino acids), and/or modified backbones.

The terms “restriction enzyme” or “restriction endonuclease” or “restrictase” may be used interchangeably and generally refers to an enzyme that cleaves double-stranded DNA into fragments at or near specific recognition sites within molecules known as restriction sites. Restriction enzymes are one class of the broader endonuclease group of enzymes. To cut DNA, all restriction enzymes make two incisions, once through each sugar-phosphate backbone (i.e. each strand) of the DNA double helix.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows a schematic of a non-limiting method for preparing a ladder composition. As be seen, provided is a non-limiting, single-stranded ladder preparation by guided cleavage of a nucleic acid (e.g., ssDNA) template. The ellipse represents a single-stranded template, which can be either circular or linear. The guided oligonucleotide (dashed lines) can be (A) pre-annealed with the template, (B) pre-assembled with a cleavage enzyme (gray triangle), or (C) mixed directly with a cleavage enzyme and the template. For all cases, the guided oligonucleotide and cleavage enzyme can bind a target region of the template, as well as cleave the template to generate different sizes of single-stranded nucleic acid fragments. Such fragments can, in turn, be optionally separated and detected by electrophoresis (e.g., displayed as a gel image or an electropherogram). The guided oligonucleotide can be identical or different sequences.

FIG. 2 shows an ssDNA ladder prepared by oligonucleotide-guided, restriction enzyme cleavage. Provided are (A) a map of restriction enzyme locations on a M13mp18 ssDNA template and (B) electropherogram analysis of ssDNA sizes by different combinations of restriction enzyme digestion. The x-axis shows fragment migration time, and the y-axis is fluorescence. Also provided is (C) a simulated gel view generated by LabChip® Reviewer of the result. The intact circular ssDNA size is 7246 nt. Enzyme (−) was used as negative control. A lower marker was used for aligning across samples.

FIG. 3 shows schematics of non-limiting guided oligonucleotides for use with restriction enzyme-based cleavage of a template (e.g., an ssDNA template). Provided is (A) a guided oligonucleotide to provide blunt ends. The guided oligonucleotide can include a target-binding region (N) that is a sufficiently complementary sequence to the target region (N) and a restriction sequence (YYYY) that is a sufficiently complementary sequence to the restriction sequence (XXXX) in the target region. Also provided is (B) a guided oligonucleotide to provide sticky or staggered ends. The guided oligonucleotide can include a target-binding region (N) that is a sufficiently complementary sequence to the target region (N) and a restriction sequence (YYYYYY) that is a sufficiently complementary sequence to the restriction sequence (XXXXXX) in the target region. Possible cut locations (dashed triangle) are provided as non-limiting examples only.

FIG. 4 shows schematics of non-limiting guided oligonucleotides for use with CRISPR-based cleavage of a template (e.g., an ssDNA template). The target region can be downstream (e.g., downstream by about 20 nt) of a reverse complement of the protospacer adjacent motif (PAM) sequence in the template. Here, this reverse complement has a sequence of CCN (from 5′ to 3′), in which PAM is then NGG (from 5′ to 3′). Provided is (A) a single guided RNA, which includes a target-binding region (N) that is a sufficiently complementary sequence to the target region (N), a CRISPR ribonucleic acid (crRNA) region, a linker, and a trans-acting CRISPR RNA (tracrRNA) region. Also provided is (B) a two-part guided RNA, which includes a guided RNA having a first portion (a target-binding region that is sufficiently complementary sequence to the target sequence region, in which this target-binding region is attached to a crRNA region) and a second portion (a tracrRNA region). The first and second portions can form a double-stranded structure by way of hybridization (dashed lines) between the crRNA region and a portion of the tracrRNA region. Possible cut locations (dashed triangle) are provided as non-limiting examples only.

FIG. 5 shows a map of M13mp18 with non-limiting restriction sites.

### DETAILED DESCRIPTION

The present disclosure encompasses methods for producing size- or mass-standard ladders, as well as compositions including such ladders. In some non-limiting embodiments, the composition can include a single-stranded nucleic acid size standard. As described herein, the ladder can be prepared by using guided cleavage of single-stranded templates. Any useful template can be employed, e.g., single-stranded templates can be from a natural resource or can be a synthetic construct or engineered construct from a natural resource (e.g., engineered M13 DNA with one or more insert, such as an insert having 1000 nucleotides). Furthermore, the template can be a linear construct or a circular construct. In some embodiments, the template (e.g., an ssDNA template) can be either linear or circular, with no limitation on size.

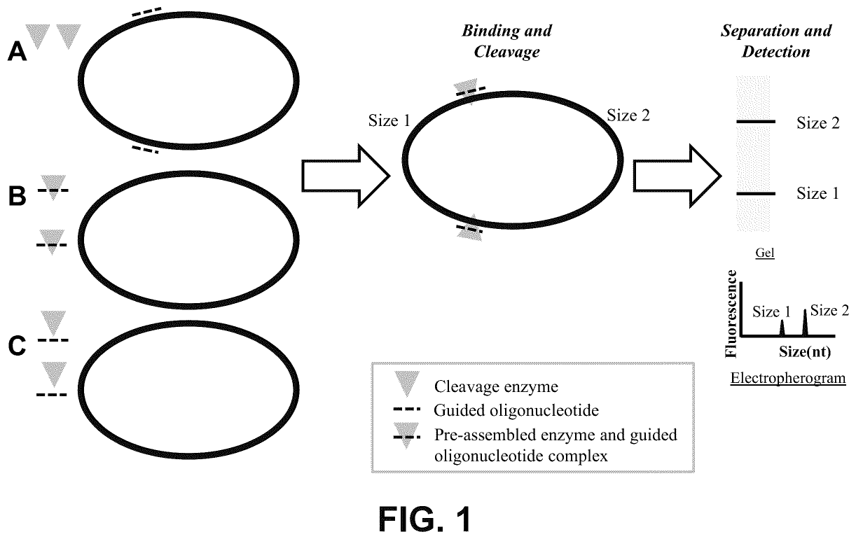

FIG. 1 provides a non-limiting method for preparing a ladder composition. Such a method can include incubating a template (ellipse) in the presence of a guided oligonucleotide (dashed lines) and a cleavage enzyme (triangle), thereby generating a ladder composition including a plurality of cleaved, single-stranded nucleic acids.

The template can be a single-stranded nucleic acid (e.g., ssDNA, ssRNA, or ssDNA/RNA). In turn, the ladder composition can include a plurality of cleaved template (e.g., cleaved ssDNA, ssRNA, or ssDNA/RNA). Furthermore, a target region within the template can include a restriction sequence, a recognition sequence, a protospacer region, a protospacer adjacent motif (PAM) sequence, or a reverse complement thereof. Such sites, when bound to the guided oligonucleotide (or guide), can provide a location within the target region to be cleaved by a cleavage enzyme.

The guided oligonucleotide can be configured to bind to the target region of the template. In one instance, the guided oligonucleotide (or guide) can include a target-binding region configured to bind (or hybridize) a target region within the template. In some embodiments, the guide includes a restriction sequence, a recognition sequence, a protospacer region, a PAM sequence, or a reverse complement thereof. The template and guide can be designed to bind (or hybridize) to each other. For instance, if the template includes a restriction sequence, then the guide can include a reverse complement of the restriction sequence to ensure hybridization between the template and the guide, thereby forming a hybridized, double-stranded structure. Such double-stranded structures, in turn, can recruit a cleavage enzyme and facilitate cleavage of a location within a target region of the template. Non-limiting examples of such sites are described herein.

In particular embodiments, the template can include a plurality of target regions, and a guide can be designed to bind (or hybridize) each target region within the template. In this way, the method can include the use of a plurality of guides, in which a first guide is configured to bind to a first target region within the template, a second guide is configured to bind to a second target region within the template, etc. In the presence of cleavage enzymes, a single template may be cleaved an n number of times that is determined by the n number of guides that can bind to the template (e.g., in which n can be 1, 2, 3, 4, 5, or more).

The cleavage enzyme can be configured to bind to the guide and to cleave a location within the target region, thereby generating the ladder composition comprising a plurality of cleaved, single-stranded nucleic acids. Non-limiting examples of cleavage enzymes are described herein.

The templates, guides, and cleavage enzymes can be provided in any useful manner. For instance, these components can be provided serially or simultaneously; or can be introduced in a manner to provide complexes. As seen in FIG. 1A, a guide can be initially provided to a template, thereby providing a complex that includes a bound template. Then, one or more cleavage enzymes can be provided to the bound template, thereby generating cleaved nucleic acids. Such cleavage products can be optionally separated and/or detected.

Other complexes can be formed. As seen in FIG. 1B, a guide can be initially provided to a cleavage enzyme, thereby providing a pre-assembled complex including a guide bound to the enzyme. Then, the template can be provided, thereby generating cleaved nucleic acids derived from the template. In yet other embodiments, components are provided separately. As seen in FIG. 1C, a guide, a cleavage enzyme, and a template can be added separately into a reaction mixture, thereby generating cleaved nucleic acids.

The guide and cleavage enzymes can be any combination to provide guided cleavage of the template. In one instance, the guide can include ssDNA, and the cleavage enzymes can include a restriction enzyme, a nicking enzyme, or an Argonaute protein. In yet another instance, the guide can include ssRNA, and the cleavage enzymes can include a Cas enzyme. In yet other embodiments, an initial enzyme that preferentially cleaves ssDNA can be modified to also cleave ssRNA, and vice versa. Furthermore, the cleavage fragments obtained from one or more methods (e.g., any described herein) can be mixed together for the final ladder preparation.

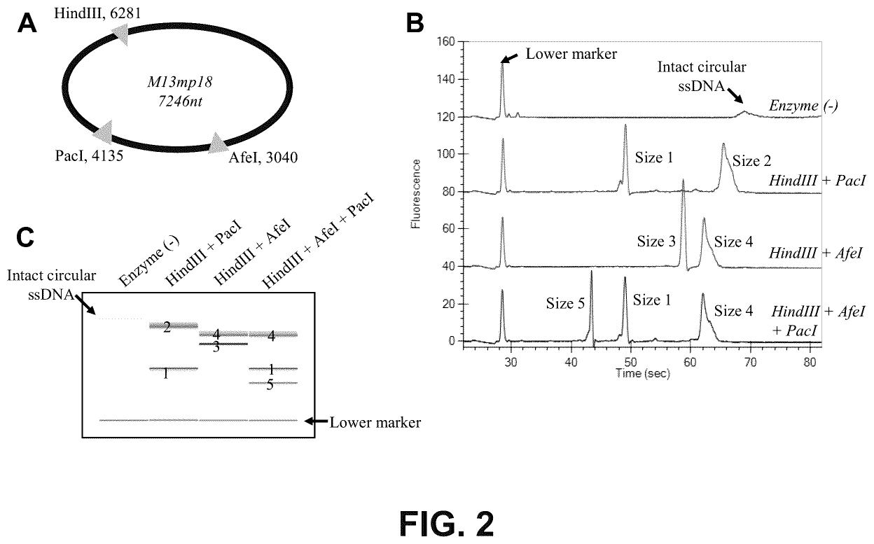

In some embodiments, the ssDNA size standard is generated by guided cleavage of an ssDNA template (see, e.g., FIG. 1). For instance, a guide can be pre-annealed with a target ssDNA template (see, e.g., FIG. 1A), pre-assembled with a cleavage enzyme before providing a target ssDNA template (e.g., as in FIG. 1B), or mixed directly with a cleavage enzyme and the ssDNA template (e.g., as in FIG. 1C). Here, the guide and cleavage enzyme can be configured to bind the target region of ssDNA template, thereby allowing the enzyme to cleave the ssDNA template to generate different size of ssDNA fragments. The size will depend on where the guide binds or hybridizes to the template, thereby providing a cut location for the cleavage enzyme. The fragments may optionally be separated and/or detected by electrophoresis (e.g., displayed as a gel image or an electropherogram in FIG. 1). Characterization of a non-limiting library preparation is provided in FIG. 2 and Example 1 herein.

Methods including particular cleavage enzymes are also described. In some embodiments, a template (e.g., an ssDNA template) can be cleaved by a restriction enzyme or a nicking enzyme together with short, single-stranded guided oligonucleotide. Restriction enzymes recognize short DNA sequences (or restriction sites) and cleave double-stranded DNA at specific sites within or adjacent to these sequences. Approximately 3,000 restriction enzymes, recognizing over 230 different DNA sequences, have been discovered. Restriction enzymes have been in bacteria, viruses, archaea, and eukaryotes. Nicking enzymes only cut one strand of duplex DNA, while leaving the other strand intact. Over 200 nicking enzymes have been studied, and some of them are available commercially and are routinely used for research and in commercial products. Any useful restriction enzymes and nicking enzymes may be used with the methods herein, and non-limiting examples of such enzymes are described herein.

In some embodiments, the template (e.g., an ssDNA template) can be cleaved by restriction enzyme or nicking enzyme together with an ssDNA as a guide. A non-limiting example is shown in Example 1. With this method, the guide can possess a minimal size (or length) that is required for restriction/nicking enzyme binding and cleavage. Non-limiting lengths for guides for use with a restriction enzyme or a nicking enzyme is from about 10 nt to 30 nt. The guide can be designed as a reverse complement strand of a target region in the template. In turn, the guide can be annealed with the template (e.g., by denaturation and annealing processes) to form a short double-stranded region, which can be recognized and cleaved by restriction/nicking enzymes. Non-limiting denaturation and annealing conditions can include any described herein, such as denaturation at an elevated temperature (e.g., denaturation at about 85° C. or higher for any useful time, such as about two minutes or more; including heating at 70° C. for 2 minutes (min) with immediate chilling on ice for about 5 min before analysis); and/or annealing at an elevated temperature with a process of cooling down to 37° C. or a lower temperature (e.g., annealing at about 90° C. for 4 min, then at 70° C. for 10 min, and finally ramped down to 37° C. at 0.1° C./sec for 20 min).

One or multiple guides (e.g., ssDNA short oligonucleotides) can be annealed with the template at the same time or in a sequential manner. One or multiple corresponding restriction/nicking enzyme can be applied for cleavage at the same time or in a sequential manner. Any commercially available or reported restriction or nicking enzymes (natural resource or engineered) can be used for a ladder preparation. After cleavage, the restriction/nicking enzyme can be inactivated by use of heat or chemical chelation (for example, by adding ethylenediaminetetraacetic acid (EDTA)).

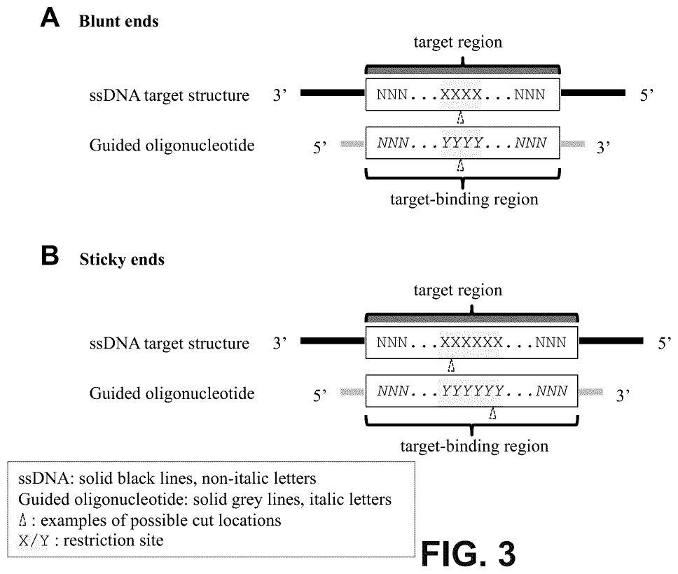

FIG. 3 provides a schematic of non-limiting guides and templates for use with restriction enzymes. As can be seen in FIG. 3A, the guide can include a target-binding region (italicized), which further includes a restriction sequence (YYYY, which corresponds to a single strand of a double-stranded restriction site). Furthermore, the target-binding region is configured to bind (or hybridize) the target region of the template. Cut locations can be located within the restriction site, in which cleavage between the two strands can provide blunt ends. Sticky or staggered ends can also be produced after cleavage, as seen in FIG. 3B.

In other aspect, the template (e.g., an ssDNA template) can be cleaved by DNA-guided, programmable endonuclease together with an ssDNA as a guide. For instance, prokaryotic Argonaute (pAgo) proteins are programmable endonucleases involved in cellular defense against foreign genetic elements. With this method, Argonaute proteins from natural resource or engineered proteins can be used for the ladder preparation. Thermus thermophilus pAgo (TtAgo) can use short, single-stranded DNA (ssDNA) guides to bind to target DNA. For use with an Argonaute protein, the guide can include a target-binding region and a recognition sequence, in which non-limiting recognition sequences for use with Argonaute proteins are described herein. One or multiple guides can be used for guided cleavage at the same time or sequentially together with programmable endonuclease proteins, buffers, and other components. Cleavage can be halted by heat inactivation or chemical inactivation (for example, by adding EDTA).

In yet other aspects, the template (e.g., an ssDNA template) can be cleaved by a Cas (clustered regularly interspaced short palindromic repeats-associated or CRISPR-associated) enzyme together with an ssRNA as a guide. The CRISPR-Cas system has been shown to provide adaptive immunity against mobile genetic elements. Double-stranded DNA cleavage by Cas9 enzyme is a hallmark of type II CRISPR-Cas immune systems. Cas9-guide RNA complexes recognize 20-bp sequences in DNA and generate a site-specific, double-strand break. Furthermore, ssDNA cleavage is an intrinsic activity of the Cas9 enzyme family that involves a distinct mode of substrate binding and catalytic domain organization. DNA-cleaving Cas enzymes (e.g., like Cas9, Cas12a, or others herein) are of great interest for genome editing. The specificity of these DNA nucleases is determined by guides, which provides great targeting adaptability. Besides this general method of programmable DNA cleavage, these nucleases have different biochemical characteristics that can be exploited for different applications.

In some embodiments, the template (e.g., an ssDNA template) can be cleaved by CRISPR-Cas enzyme together with an ssRNA as a guide. With this method, DNA-cleaving Cas enzymes, for example, but not limited to, Cas9 and Cas12a proteins from natural resource or engineered proteins, can be used for the ladder preparation. The design of RNA guides is based on the specific sequence of the target region (in the template) and corresponding requirements from Cas enzymes. One or multiple ssRNA guides can be used for guided cleavage at the same time or sequentially together with CRISPR-Cas enzymes, buffers, and the like. The CRISPR-Cas enzyme cleavage can be halted by heat inactivation or chemical inactivation (for example, by adding EDTA).

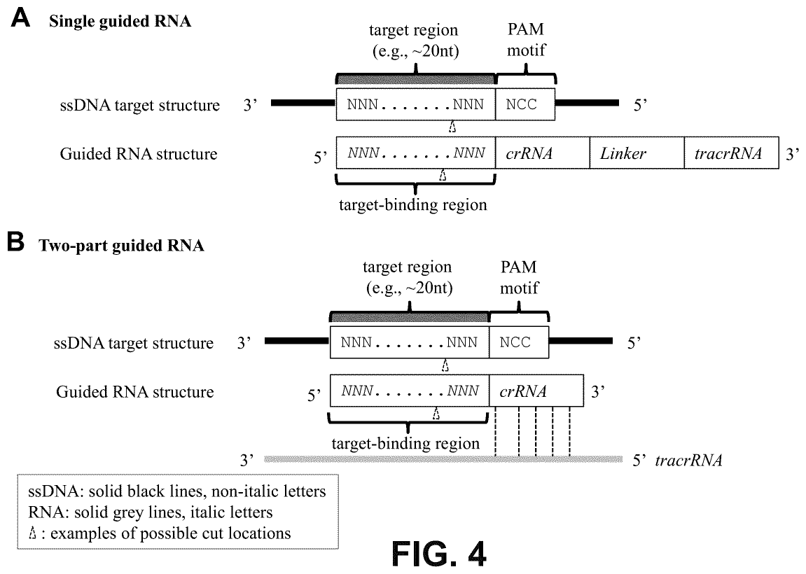

FIG. 4 provides a schematic of non-limiting guides and templates for use with Cas enzymes. As can be seen in FIG. 4A, the guide can include a target-binding region (italicized), a CRISPR ribonucleic acid (crRNA) region, an optional linker, and a trans-acting CRISPR RNA (tracrRNA) region. The sequence in PAM motif is related to the corresponding Cas enzyme. Furthermore, the target-binding region is configured to bind (or hybridize) the target region of the template. Cut locations can be located within the target region and the target-binding region. In some embodiments, the Cas enzyme can be modified to provide nickase activity.

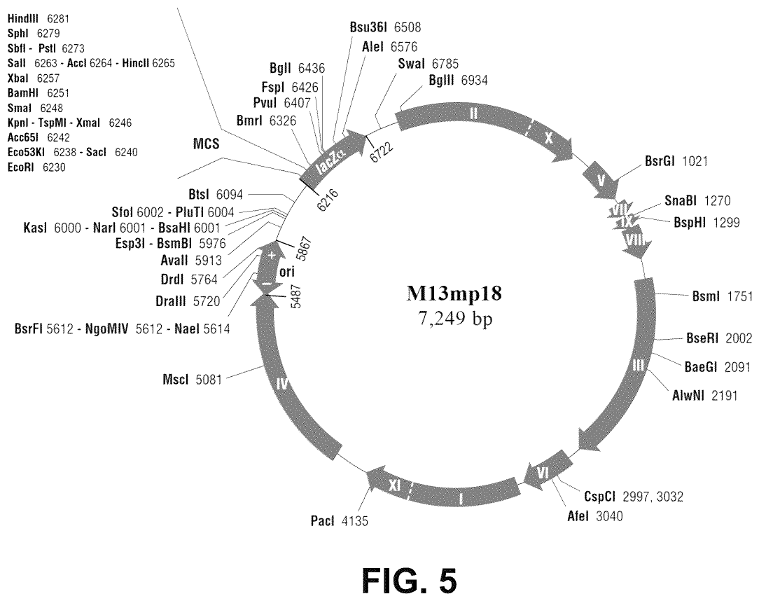

In particular embodiments, the ladder can be prepared from one or multiple methods described herein. Such methods can be used to separately prepare populations of cleaved nucleic acids, and then such populations can be mixed together as final product. In one non-limiting embodiment, the ssDNA template is M13mp18 (a single-stranded, circular DNA having about 7.2 kilobases (kb), see, e.g., FIG. 5). Such a template can be used to generate the following linear size standard:
a population including at least one cleaved nucleic acid having 7.2 kb (e.g., by cleavage with one restriction enzyme (e.g., HindIII) to linearize the initial template that is circular);
a population including a first cleaved nucleic acid having 2.1 kb and a second cleaved nucleic acid having 5.1 kb (e.g., by cleavage with two restriction enzymes (e.g., HindIII and PacI));
a population including a first cleaved nucleic acid having 3.2 kb and a second cleaved nucleic acid having 4 kb (e.g., by cleavage with two restriction enzymes (e.g., HindIII and AfeI)); and/or
a population including a first cleaved nucleic acid having 1.0 kb and a first cleaved nucleic acid having 6.2 kb (e.g., by cleavage by CRISPR-Cas enzymes).
   In some embodiments, a ladder composition can be prepared by mixing the above fragments to generate the following size standard: about 1.0 kb, 2.1 kb, 3.2 kb, 4 kb, 5.1 kb, 6.2 kb, and 7.2 kb. In other embodiments, a ladder composition can be prepared by mixing the above fragments to generate the following size standard: about 1.1 kb, 2.1 kb, 3.2 kb, 4 kb, 5.1 kb, 6.2 kb, and 7.2 kb.

In some embodiments, a size standard (e.g., a ssDNA size standard) prepared by the methods herein can be separated and detected in an electrophoresis platform, for example, but not limited to, in agarose or polyacrylamide gels, capillary electrophoresis, microfluidic chip, high performance liquid chromatography, mass spectrometry, and the like.

In some embodiments, the methods herein allow for preparation of ladder compositions with high efficiency. Without wishing to be limited by mechanism, the present methods employ a reduced number of reagents (e.g., short guided oligonucleotides and cleavage enzymes) having a reduced number of reaction events (e.g., binding and cleaving events). By selecting process conditions that facilitate effective binding and cleaving, ladders can be produced in an efficient manner in terms of cost or process. Furthermore, as described herein, produced ladder preparations may not require purification and/or dilution prior to use, thereby avoiding post-processing operations.

In some embodiments, the methods herein allow for preparation of ladder compositions having nucleic acids of increased size. For example, PCR can be used to prepare nucleic acids for use as ladders, yet longer nucleic acids are difficult to prepare using PCR. The methods herein can be used to prepare nucleic acids having a length that is 500 bases or longer. Further non-limiting lengths can include more than about 500 bases (0.5 kilobases or kb), 1000 bases (1 kb), 2000 bases (2 kb), 3000 bases (3 kb), 4000 bases (4 kb), 5000 bases (5 kb), 6000 bases (6 kb), 7000 bases (7 kb), or 8000 bases (8 kb). In other embodiments, lengths can include from about 0.5 to 3 kb, 0.5 to 5 kb, 0.5 to 10 kb, 0.5 to 20 kb, 0.5 to 50 kb, 0.5 to 100 kb, 0.5 to 200 kb, 0.5 to 500 kb, 0.5 to 750 kb, 1 to 3 kb, 1 to 5 kb, 1 to 10 kb, 1 to 20 kb, 1 to 50 kb, 1 to 100 kb, 1 to 200 kb, 1 to 500 kb, 1 to 750 kb, 2 to 3 kb, 2 to 5 kb, 2 to 10 kb, 2 to 20 kb, 2 to 50 kb, 2 to 100 kb, 2 to 200 kb, 2 to 500 kb, 2 to 750 kb, 3 to 5 kb, 3 to 10 kb, 3 to 20 kb, 3 to 50 kb, 3 to 100 kb, 3 to 200 kb, 3 to 500 kb, 3 to 750 kb, 5 to 10 kb, 5 to 20 kb, 5 to 50 kb, 5 to 100 kb, 5 to 200 kb, 5 to 500 kb, 5 to 750 kb, 10 to 20 kb, 10 to 50 kb, 10 to 100 kb, 10 to 200 kb, 10 to 500 kb, or 10 to 750 kb).

In other embodiments, the methods herein allow for preparation of ladder compositions that possess high purity. For example and without limitation, the method herein can employ a single-stranded template to produce cleaved, single-stranded nucleic acids. By starting with a highly pure template, cleaved nucleic acids with high purity can be obtained with requiring further purification steps. The methods herein can also be adapted to avoid components that would reduce the purity of the generated ladder composition. For example, as PCR or other amplification reactions are avoided, reagents to promote amplification (e.g., polymerases, added salts, added buffers and buffer components, and the like) can be avoided. In another example, cleavage of the template can be adapted to avoid excess or added components (e.g., bovine serum albumin (BSA), denaturants, and the like).

Purity can be determined in any useful manner. In one non-limiting instance, purity can be based the homogeneity of the single-stranded fragment (e.g., homogeneity as assessed by the fragment size or fragment sequence). In particular embodiments, homogeneity can depend on the efficiency of the enzymatic cleavage reaction. In another instance, purity of the buffer in the ladder composition can be assessed. For example, the buffer in the final ladder composition can be identified as being useful for long-term storage (e.g., storage for one month, 3 months, 6 months, one year, or longer without damaging single-stranded nucleic acids or otherwise influencing other properties of the nucleic acids, such as by the presence of undesired double-stranded structures, undesired fragment sizes, or the like). Yet in some instances, double-stranded structures may be formed within the buffer, and the ladder composition can be denatured prior to use. For example and without limitation, the ladder and test sample can be exposed to a denaturation process (e.g., any described herein) before analysis, e.g., such as before electrophoresis. In another example, the buffer in the final ladder composition can be identified as not interfering with one or more down-stream processes (e.g., assays, purification processes, separation processes, and the like, such as electrophoresis).

Methods of determining purity can include any useful methodology, including but not limited to use of spectrophotometers, electrophoresis analyzers, ssDNA assays, and the like. Non-limiting examples include analysis of various absorbance measurements (e.g. absorbance at 260 nm (A260), absorbance at 280 nm (A280), or a ratio of A260 and A280 such as with NanoDrop™ Spectrophotometer from Thermo Fisher Scientific, Inc., Waltham, MA); amounts of ssDNA (e.g., such as with Qubit™ ssDNA Assay Kit from Thermo Fisher Scientific); amount of dsDNA (e.g., such as with Qubit™ dsDNA HS Assay Kit); and fragment analysis by use of electrophoresis (e.g., such as with LabChip® from PerkinElmer, Inc., Waltham, MA).

In some embodiments, conditions during cleavage can be optimized to be compatible with downstream electrophoresis methods without the need of post-cleavage purification. One such condition can include the presence of a blocking agent, such as bovine serum albumin (BSA) or recombinant albumin (rAlbumin). Typically, BSA or rAlbumin is a standard component in buffers for use during cleavage by restriction or nicking enzymes. In use, albumin-containing components can be used to stabilize proteins in reaction mixtures and/or to prevent adhesion of reaction products or reagents (e.g., primers, proteins, and such) to surfaces of reaction tubes or pipettes. When such a buffer is used, the cleaved products (e.g., as an ssDNA ladder) can be used directly for agarose gel or PAGE gel analysis. However, such products may need to be diluted or purified prior to conducting electrophoretic analysis (e.g., on a LabChip® platform, which has the limitation of BSA concentration of up to 0.05 mg/mL). By employing the methods herein, such blocking agent(s) can be avoided during cleavage.

In the cleavage reaction for ladder production, the template (e.g., ssDNA) and enzymes are typically provided in sufficient amounts, thereby minimizing the risk of loss performance due to surface binding. Therefore, in some embodiments, the cleavage buffer can be modified to remove or minimize the amount of BSA or rAlbumin. Thus, resultant fragments (e.g., cleaved ssDNA fragments) from enzyme cleavage can be applied for electrophoresis without the need of purification and/or dilution.

In another example, as the presence of double-stranded (ds) nucleic acid may be undesired, the present method avoids excess presence of such ds nucleic acids. To the extent that double-stranded structures are present upon binding of the guided oligonucleotide to the template, such structures can be designed to have minimal effect on the generated ladder. For instance, shortened guided oligonucleotides (or guides) can be employed, such that the length of the guides can be 10, 20, 30, 40 nt, or greater times shorter than the cleaved nucleic acids that are provided in the ladder composition.

In yet another example, the methods herein allow for preparation of ladder compositions that possess predominantly single-stranded and cleaved nucleic acids. As described herein, the methods herein can avoid continuous formation of double-stranded structures (e.g., as in PCR) to provide cleaved, single-stranded nucleic acids. Thus, the resultant cleaved nucleic acids can be considered to be single-stranded. The extent of single-strandedness can be determined in any useful manner, e.g., by measuring absorption at 260 nm, in which hyperchromicity results in increased absorbance upon denaturation of double-stranded structures; by use of intercalating dyes, which can bind to double-stranded structures, and the like. The extent of single-strandedness can be determined for a population of cleaved, single-stranded nucleic acids, in which at least 80%, 90%, or more of the population can be single-stranded.

In yet other embodiments, the present disclosure can be employed to resolve current limitations for ssDNA size standard preparation, especially for large-sized ladders (e.g., having more than 0.5 kb, 1 kb, 2 kb, or larger). Such large-sized ladders may be useful for applications that analyze longer fragments of nucleic acids. Applications can include, e.g., gene therapies, mRNA-based vaccines, or other therapeutic solutions. In particular embodiments, the ladder composition can include one or more of the following characteristics:
The starting template can be a natural resource of single-stranded nucleic acid (e.g., ssDNA). In non-limiting embodiments, the template can be commercial available at large-scale with low cost.
The desired size of cleaved nucleic acids can be generated by sequence-specific cleavage. In theory, there is no limitation to a required standard size.
The cleaved nucleic acids can be used directly without the need of additional purification, for example, to remove one strand from a double-stranded template or a double-stranded cleavage product. Such an approach can simplify the production process and reduce the cost of production.
The cleavage methods herein can be scaled-up in a large container because the cleavage temperature can be conducted at a single temperature, as compared to PCR which can require thermocycling. By avoiding thermal ramping and PCR reagents, the production process can be simplified.

For any methods herein, benefits can include control of the cleaved ladder size by the user or by intended use, which is not limited by the availability of commercially available cleavage enzymes.

In some embodiments, the present disclosure encompasses methods for single-stranded nucleic acid (e.g., ssDNA) ladder compositions, which is independent from PCR, isothermal amplification that can generate double-stranded products, or DNAzyme cleavage. In particular embodiments, such methods can allow for large-scale production.

In other embodiments, the ladder composition can include a first population of first cleaved, single-stranded nucleic acids. The population can possess any characteristic. In one instance, the population can include nucleic acids having a particular length. In some embodiments, at least 50%, 60%, 70%, 80%, or more of the population has a length of 0.5, 1, 2, 3, 4, 5, 6, 7, 8, 9, 10, or more kb; or a length from about 0.5 to 750 kb (e.g., from 0.5 to 3 kb, 0.5 to 5 kb, 0.5 to 10 kb, 0.5 to 20 kb, 0.5 to 50 kb, 0.5 to 100 kb, 0.5 to 200 kb, 0.5 to 500 kb, 1 to 3 kb, 1 to 5 kb, 1 to 10 kb, 1 to 20 kb, 1 to 50 kb, 1 to 100 kb, 1 to 200 kb, 1 to 500 kb, 1 to 750 kb, 2 to 3 kb, 2 to 5 kb, 2 to 10 kb, 2 to 20 kb, 2 to 50 kb, 2 to 100 kb, 2 to 200 kb, 2 to 500 kb, 2 to 750 kb, 3 to 5 kb, 3 to 10 kb, 3 to 20 kb, 3 to 50 kb, 3 to 100 kb, 3 to 200 kb, 3 to 500 kb, 3 to 750 kb, 5 to 10 kb, 5 to 20 kb, 5 to 50 kb, 5 to 100 kb, 5 to 200 kb, 5 to 500 kb, 5 to 750 kb, 10 to 20 kb, 10 to 50 kb, 10 to 100 kb, 10 to 200 kb, 10 to 500 kb, or 10 to 750 kb). In other embodiments, a single population can have a plurality of first cleaved, single-stranded nucleic acids and a plurality of second cleaved, single-stranded nucleic acids; in which the first cleaved, single-stranded nucleic acids has a first length; and in which the second cleaved, single-stranded nucleic acids has a second length. The first and second lengths can be different.

In some embodiments, the ladder composition can include a first population and a second population, in which each population includes a plurality of cleaved, single-stranded nucleic acids. In other embodiments, the ladder composition includes a plurality of populations; and each population can include a respective plurality of cleaved, single-stranded nucleic acids.

Furthermore, in some embodiments, each population can be different from another population. Differences between populations can include, e.g., differences in size, mass, and/or sequence. Such differences between populations can be characterized by a certain percentage of each population having a certain characteristic. For example and without limitation, at least 90% of the first population can have a length that is longer than at least 90% of the second population.

In other embodiments, each population can include a shared characteristic. For example and without limitation, at least 80%, 90%, or more of a population (e.g., or each population within a plurality of populations) can be single-stranded. In another example, at least 50%, 60%, 70%, 80%, 90%, or more of a population (e.g., or each population within a plurality of populations) has a length of 0.5, 1, 2, 3, 4, 5, 6, 7, 8, 9, 10, or more kb; or a length from about 0.5 to 750 kb (e.g., any ranges described herein).

When a plurality of populations is present, each population can have a shared characteristics (e.g., a length from about 0.5 to 750 kb). Yet, each population can still possess a different characteristic (e.g., a first population having a length of about 0.5 kb, a second population having a length about 1 kb, a third population having a length about 2 kb, and the like). A skilled artisan would understand how desired characteristics of such ladder compositions and populations within a composition can be determined, as well as how methods herein can be designed to be prepare such compositions.

The ladder composition can include any other further characteristics, such as those described in U.S. Pat. No. 8,273,863; U.S. Pat. Pub. Nos. US 2019/0203242 or US 2007/0178482; Int. Pub. No. WO 2020/232286; Bush et al., Molecules 2020 Jul. 26; 25(15):3386; and/or Gu et al., Biotechniques 2013 June; 54(6):337-43, each of which is incorporated herein by reference in its entirety.

### Templates

Any useful template can be employed. In some embodiments, the template can be obtained from natural sources (e.g., bacteriophage, viral nucleic acid, adeno-associated viral nucleic acid, and the like). Non-limiting examples of templates (e.g., an ssDNA template) include an M13 bacteriophage, such as M13mp18 (a single-stranded, circular DNA having about 7.2 kb); a single-stranded viral DNA isolated from a Φ174 bacteriophage, such as Φ174 Virion DNA (a single-stranded, circular DNA having about 5.4 kb); or a single-stranded, linear adeno-associated virus (AAV) DNA, such as AAV serotype 1, 2, 4, or 7-11 (a single-stranded, linear DNA having about 4.7 to 4.8 kb). Such templates can be obtained from any useful bacteriophage or virus, in which such template can include DNA and/or RNA.

In some embodiments, the template (e.g., ssDNA template) can be genetically engineered forms of nucleic acid sequences obtained from natural resources. In other embodiments, the template (e.g., ssDNA template) includes a synthetic construct.

In some embodiments, the template (e.g., ssDNA template) can be prepared from an in vitro process including, for example, but not limited to, production of ssDNA from double-stranded plasmids in vitro. A non-limiting example of an in vitro process is provided in, e.g., Thermo Fisher Scientific, “Production of Single-stranded Circular DNA Molecules from Supercoiled Double-stranded Plasmids in vitro,” 2012, 1 page (accessible at tools.thermofisher.com/content/sfs/brochures/76-circular-ssdna-from-supercoiled-double-stranded-plasmids-in-vitro.pdf), which is incorporated herein by reference in its entirety. In other embodiments, the template (e.g., ssDNA template) can be in vitro amplified products from either natural resources or synthetic constructs or an in vitro process. The amplification method can be, but not limited to, rolling circle amplification (RCA).

### Guided Oligonucleotides

Guided oligonucleotides (or guides) can be single-stranded structures formed from DNA, RNA, or both. In particular embodiments, the guide is a synthetic construct. Based on desired and known target regions in the template, a guide can be designed to include a target-binding region that is configured to bind (or hybridize) a target region within the template. Furthermore, the guide can include a region to bind (or hybridize) to a portion of the cleavage enzyme. Upon binding the guide-bound template, the cleavage enzyme can cleave the template (e.g., within the target region of the template). In this way, cleavage of the template is directed by the guide.

The target-binding region of the guide can include one or more restriction sequences, recognition sequences, protospacer regions, PAM sequences, or a reverse complement thereof. Each of these restriction sequences, recognition sequences, protospacer regions, PAM sequences, or reverse complements thereof can be a single-stranded structure. Upon binding of such sequences to the template, a double-stranded structure is formed, and such double-stranded structures form the site or motif that is bound by the cleavable enzyme. Thus, a guide can include a strand of any restriction motif described, any recognition motif described herein, or reverse complements of any of these.

Accordingly, a single-stranded sequence from any site or motif herein can be provided within the guide. For instance, when the cleavage enzyme is a restriction enzyme, the guide can include a restriction sequence from a corresponding restriction motif. When the restriction sequence of the guide binds the target region of a template, a double-stranded restriction motif can be formed. Then, such a motif can bind the desired restriction enzyme.

A skilled artisan would be able to develop a guide to facilitate binding to and cleavage of the template. For example and without limitation, the following design considerations can be employed:

Step 1: Identify restriction/nicking enzymes based on the sequence of the template and target regions within the template. For an ssDNA template, locations of restriction enzymes can be identified using commercial or free software (e.g., NEB cutter 2.0, as provided in nc2.neb.com/NEBcutter2/). A non-limiting example of a sequence map is provided in FIG. 5.

Step 2: Choose a proper enzyme or a group of enzymes (e.g., a plurality of enzymes). Factors for consideration in choosing restriction/nicking enzymes (e.g., any described below or in FIG. 5) include one or more of the following:
The cleaved product size meets the needs (e.g., size needs);
Enzyme cost. For example and without limitation, if only one enzyme is selected for single cleavage of M13mp18, then HindIII can be selected as it is much cheaper than other enzymes; and/or
If multiple enzymes are used for cleavage at the same time, then the cleavage buffer should be compatible for such different enzymes. A non-limiting example of software for determining buffer compatibility includes, e.g., nebcloner.neb.com/#!/redigest.

Step 3: Design a guide based on the selection of restriction/nicking enzymes. Factors for consideration in designing the guide include one or more of the following:
Including the sequences (for target-binding regions) that can bind (complement or hybridize) to the target restriction/nicking site;
Including additional sequences, e.g., 6 nt added on either side of the restriction/nicking site (see, e.g., www.neb.com/tools-and-resources/usage-guidelines/cleavage-close-to-the-end-of-dna-fragments), in which such additional sequence can be configured to bind (complement or hybridize) to the target region of the template; and/or
Consider use of standard desalt oligonucleotides, which can reduce the cost of manufacture.

Non-limiting examples of guides are provided in Table 1 of Example 1 (restriction sequences are highlighted in bold).

Guides for other cleavage enzymes can be designed to include target-binding regions and other restriction sequences (e.g., for use with a nicking enzyme), recognition sequences (e.g., for use with an Argonaute protein), protospacer regions (e.g., for use with a Cas enzyme), PAM sequences (e.g., for use with a Cas enzyme), or a reverse complement thereof.

In particular, guides for use with Cas enzymes can include a single-stranded guide or a double-stranded guide (a two-part guide). Typically, a Cas enzyme is recruited to the target by two RNA regions: a tracrRNA region and a crRNA region. A synthetic guide can be used to provide these two regions in a contiguous, single nucleic acid. Thus, in some embodiments, the single-stranded guide includes a target-binding region, a crRNA region, and a tracrRNA region. Optionally, a linker can be present between the crRNA and tracrRNA regions. In other embodiments, the guide is a double-stranded guide, which includes a first strand having a target-binding region and a cRNA region, as well as second strand having a tracrRNA region. The first and second strands, together, can form a double-stranded structure.

A skilled artisan would be able to develop a guide to facilitate binding to and cleavage of the template by using a Cas enzyme. For example and without limitation, the following design considerations can be employed (which can be implemented manually or using software, including free or commercial solutions):

Step 1: Identify candidate sequences of reverse complement PAM sequence in the ssDNA template. The PAM sequence, in general, is “NGG”; and other PAM sequences are described herein. It can be other formats that is CRISPR enzyme-dependent. The template (e.g., an ssDNA template) can include a reverse complement of the PAM sequence “NGG”, which is “CCN” (from 5′ to 3′).

Step 2: Choose candidate PAM locations in the template based on the desired cleavage product size.

Step 3: Design a guide based on the selected PAM sequence. In some embodiments, the target region is around 20 nt downstream of the 5′-CCN sequence in the template. Non-limiting examples for designing guides are provided in, e.g., takarabio.com/learning-centers/gene-function/gene-editing/gene-editing-tools-and-information/how-to-design-sgrna-sequences; snapgene.com/guides/design-grna-for-crispr; or idtdna.com/pages/community/blog/post/guide-rna-design-be-on-target!, the disclosure of each of which is incorporated herein by reference in its entirety.

Non-limiting examples of guide designs are shown in FIG. 4. As seen in FIG. 4A, the guide can include a single guide RNA, which can include the following (from 5′ end to 3′ end):
a target-binding region including a complementary sequence to the target region in the ssDNA template (e.g., which can optionally be about 20 nt);
a crRNA sequence;
a linker sequence that links the crRNA and tracrRNA (e.g., any linker described herein); and
a tracrRNA sequence.

FIG. 4B shows a two-part guide, which includes a first portion and a second portion. The first portion can include a nucleic acid (e.g., RNA) having the following:
a target-binding region including a complementary sequence to the target region in the ssDNA template (e.g., which can optionally be about 20 nt); and
a crRNA sequence.

The second portion can include a nucleic acid (e.g., RNA) having a tracrRNA sequence. A portion of the tracrRNA sequence can be complementary to the crRNA sequence, as indicated by dashed vertical lines. The guide (e.g., at the RNA 5′ end and/or 3′ end) can include one or more nucleotide modifications, for example, but not limited to, 2′OMe bases, phosphonothioate or phosphorothioate bond modifications, and the like. Further modified nucleotides are described herein.

### Cleavage Enzymes

Any useful cleavage enzyme may be employed. Cleavage enzymes can include one or more restriction enzymes, nicking enzymes, programmable endonucleases, Argonaute proteins, Cas enzymes, any useful nuclease (e.g., an endonuclease), or variants thereof.

In one embodiment, the cleavage enzyme is a restriction enzyme, which can include blunt end and/or sticky end restriction endonuclease. Such an enzyme can be used with a guided oligonucleotide having a restriction sequence. In some embodiments, the restriction sequence has a length of about 4 nt to 10 nt. Examples of non-limiting restriction sequences include 5′-AAGCTT-3′ (e.g., for HindIII), 5′-TTAATTAA-3′ (e.g., for PacI), 5′-AGCGCT-3′ (e.g., for AfeI), or a reverse complement thereof. A skilled artisan would be able to determine a restriction site and corresponding restriction sequences for a particular restriction enzyme, as well as to develop a guided oligonucleotide to facilitate binding to and cleavage of the template.

Non-limiting examples of restrictions enzymes can include one or more of the following: Acc65I, AccI, AfeI, AleI, AlwNI, AvalI, BaeGI, BamHI, BglI, BglII, BmrI, BsaHI, BseRI, BsmBI, BsmI, BspHI, BsrFI, BsrGI, Bsu36I, BtsI, CspCI, DraIII, DrdI, Eco53KI, EcoRI, Esp3I, FspI, HincII, HindIII, KasI, KpnI, MscI, NarI, NgoMIV, NaeI, PacI, PstI, PvuI, SacI, SalI, SbfJ, SfoI, SmaI, SnaBI, SphI, SwaI, TspMI, PluTI, XbaI, XmaI, and the like.

Yet other enzymes include AluI, AvaI, BamHI, BanII, BglII, ClaI, DraI, Eco47 III, EcoRI, EcoRV, FspI, HindIII, HpaI, HpaII, KpnI, MscI, MseI, NcoI, NdeI, NotI, NruI, PstI, PvuI, PvuII, RsaI, ScaI, SmaI, SspI, SstI, StuI, ThaI, XbaI, or XhoI. Yet other enzymes include: AatII, AbsI(x), AcuI, AflII, AgeI, AhdI, AjuI(x), ApaI, ApaLI, AscI, AsiSI, AvrII, BbsI, BcgI, BciVI, BclI, BlpI, BmgBI, BmtI, BplI(x), BsaI, BsgI, BsiWI, BspEI, BspQI, BssHII, BssSI, BstAPI, BstBI, BstEII, BstXI, BstZ17I, EagI, EcoNI, EcoO109I, EcoRV, FseI, FspAI(x), HpaI, KflI(x), MauBI(x), MfeI, MluI, MreI(x), MteI(x), NcoI, NheI, NmeAIII, NotI, NruI, NsiI, PaeR7I, PaqCI, PasI(x), PflFI, PflMI, PfoI(x), PmeI, PmlI, PpuMI, PshAI, PspOMI, PspXI, PsrI(x), RsrII, SacII, SanDI, SapI, ScaI, SexAl, SfiI, SgrAI, SgrDI(x), SpeI, Srfl(x), StuI, StyI, Tth111I, XcmI, XhoI, ZraI, and the like. A skilled artisan would understand how to identify restriction sites and corresponding restriction sequences for such enzymes, as well as how to use such sites to provide cut locations within the target region of the template and/or the guided oligonucleotide.

The restriction enzymes described herein, and others that may be equivalently used in the present disclosure, are available commercially, for example from Thermo Fisher Scientific, Inc. (Waltham, MA) or New England Biolabs (Ipswich, MA). For example, although specific restriction endonucleases are herein, it will be recognized that isoschizomers, i.e., enzymes that have the same recognition site but cut in a different fashion, can be substituted and the same result will be achieved. See also Roberts, Nucl. Acids Res. 1989; 17(Suppl.):r347-r387 for other examples of restriction enzymes and their cleavage sites, which is incorporated herein by reference in its entirety.

The cleavage enzyme can be nicking enzyme (e.g., a nicking endonuclease). Such nicking enzymes can be used to cut only one strand of the double-stranded structure. For example, the guided oligonucleotide can be configured to bind to the target region of the template, thereby coordinating guided-based cleavage. Yet, cleavage by the nicking enzyme can occur only to the template strand. Such an enzyme can be used with a guided oligonucleotide having a restriction sequence. In some embodiments, the restriction sequence has a length of about 4 nt to 12 nt. Examples of non-limiting restriction sequences include 5′-NNNNNGACTC-3′ (e.g., for Nt.BstNBI) or 5′-GCAGTGNN-3′ (e.g., for Nb.BtsI), in which N can be any nucleic acid (e.g., G, C, A, T, or U, or a modified form thereof). A skilled artisan would be able to determine a restriction site and corresponding restriction sequences for a particular nicking enzyme (e.g., a top-strand specific or bottom-strand specific cleavage enzyme), as well as to develop a guided oligonucleotide to facilitate binding to and cleavage of the template.

Non-limiting examples of nicking enzymes include Nb.BbvCI, Nb.Bpu10I, Nb.BsmI, Nb.BsrDI, Nb.BssSI, Nb.BtsI, Nb.Mva1269I, Nt.AlwI, Nt.BbvCI, Nt.Bpu101, Nt.BsmAI, Nt.BspQI, Nt.BstNBI, Nt.CviPII, and the like. In other embodiments, the nicking enzyme can be a restriction enzyme (e.g., any described herein) that has been modified to nick the bottom or top strand.

The cleavage enzyme can be a programmable endonuclease. In one embodiment, the programmable endonuclease is an enzyme that recognizes user-defined nucleic acid sequences (e.g., in a template or a guided oligonucleotide), in which recognition sites can be much larger than an average restriction site of a restriction enzyme (e.g., a recognition site for the programmable endonuclease can include from about 15 base pairs (bp) to 25 bp). In some embodiments, the recognition sequence has a length of about 15 nt to 25 nt. Programmable endonucleases such as RNA-guided CRISPR-based systems and DNA-guided prokaryotic Argonautes are found throughout the prokaryotic kingdom and may differ significantly in activity and specificity.

In some embodiments, the cleavage enzyme can be an Argonaute protein. In particular embodiments, Argonaute proteins can be used for DNA-guided or RNA-guided programmable cleaving of a template. Such an enzyme can be used with a guided oligonucleotide (e.g., a guided DNA) having a recognition sequence or a reverse complement thereof. Examples of non-limiting recognition sequences include 5′-TTACCGCTAATGGT GTG-3′ (SEQ ID NO:1) or 5′-TNNNNNNNNNNXNNNNN-3′, wherein X is G, C, T, or U; N is any nucleic acid (e.g., G, C, A, T, or U, or a modified form thereof); and optionally wherein GC content is from about 10% to 70% or from 20% to 60%. The guided oligonucleotide can include the recognition sequence, and the template can be configured to bind (or hybridize) to the recognition sequence. In some embodiments, the cut location in the template can be located in the target region corresponding to, e.g., between bases 10 and 11 of the guided oligonucleotide. A skilled artisan would be able to determine a restriction site and corresponding restriction sequences for a particular Argonaute protein, as well as to develop a guided oligonucleotide to facilitate binding to and cleavage of the template. Methods and compositions for RNA-guided programmable cleaving of a template are described in Doxzen K W and Doudna J A, “DNA recognition by an RNA-guided bacterial Argonaute,” PLoS ONE 12(5):e0177097 (14 pages), which is incorporated herein by reference in its entirety.

Non-limiting examples of Argonaute proteins include Argonaute RISC catalytic component 2 (Ago2), prokaryote Argonaute (pAgo), Thermus thermophilus Argonaute (TtAgo), Methanocaldococcus jannaschii Argonaute (MjAgo), or Pyrococcus furiosus Argonaute (PfAgo).

In other embodiments, the cleavage enzyme can be a Cas enzyme. In particular embodiments, Cas enzymes can be used for RNA-guided programmable cleaving of a template. Such an enzyme can be used with a guided oligonucleotide (e.g., a guided RNA) having a protospacer adjacent motif (PAM) sequence or a reverse complement thereof. Examples of non-limiting PAM sequences include 5′-NGG, as well as 5′-CCN as a reverse complement. Yet other examples of PAM sequences include the following: 5′-NGCG, 5′-NGAG, 5′-NGNG, 5′-NAGN, 5′-NG, 5′-GAA, 5′-GAT, 5′-NNGRRT, 5′-NNGRR(N), 5′-TTTV, 5′-TYCV, 5′-TATV, 5′-NNNNRYAC, 5′-NNNNGATT, 5′-NNAGAAW, 5′-NAAAAC, as well as reverse complements of any of these. For any of these sequences, each N can be independently G, C, A, T, or U, as well as a modified form thereof.

In the guided oligonucleotide, the PAM sequence can be located about 2 nt to 6 nt downstream of the cut location. The reverse complement of the PAM sequence can be located about 2 nt to 6 nt upstream of the target region in the template. A skilled artisan would be able to determine PAM sequences and locations for these sequences for a particular Cas enzyme, as well as to develop a guided oligonucleotide to facilitate binding to and cleavage of the template.

Non-limiting examples of Cas enzymes include Cas1, Cas1B, Cas2, Cas3, Cas4, Cas5, Cas6, Cas7, Cas8, Cas9 (also known as Csn1 and Csx12), Cas10, Cas12a (Cpf1), Cas12b (C2c1), Cas12c (C2c3), Cas12d (CasY), Cas12e (CasX), Cas12f (Cas14), Cas12g, Cas12h, Cas12i, Cas12j (Cas(D), Cas12k (C2c5), Cas13a (C2c2), Cas13b, Cas13c, Cas13d, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of Streptococcus pyogenes Cas9 protein may be found in the SwissProt database under accession number Q99ZW2. In some embodiments, the unmodified Cas enzyme has DNA cleavage activity, such as Cas9. In some embodiments, the Cas enzyme is Cas9 and may be Cas9 from Streptococcus pyogenes (e.g., UniProtKB Acc. No. Q99ZW2), Streptococcus thermophilus (e.g., UniProtKB Acc. No. G3ECR1), Streptococcus pneumoniae (e.g., UniProtKB Acc. No. A0A111NJ61), Staphylococcus aureus (e.g., UniProtKB Acc. No. J7RUA5), Neisseria meningitidis (e.g., UniProtKB Acc. No. A1IQ68), Campylobacter jejuni (e.g., UniProtKB Acc. No. Q0P897), Rhodopseudomonas palustris (e.g., UniProtKB Acc. No. Q13CC2), Rhodospirillum rubrum (e.g., UniProtKB Acc. No. Q2RX87), Actinomyces naeslundii (e.g., UniProtKB Acc. No. J3F2B0), Francisella tularensis subsp. novicida (e.g., UniProtKB Acc. No. A0Q5Y3), or Corynebacterium diphtheriae (e.g., UniProtKB Acc. No. Q6NKI3). In some embodiments, the Cas enzyme directs cleavage of one or both strands at the location of a target region, such as within the target region and/or within the complement of the target region. In some embodiments, the Cas enzyme directs cleavage of one or both strands within about 1, 2, 3, 4, 5, 6, 7, 8, 9, 10, 15, 20, 25, 50, 100, 200, 500, or more base pairs from the first or last nucleotide of a target region.

The nuclease may be a Cas9 homolog or ortholog. In some embodiments, the nuclease directs cleavage of one or two strands at the location of the target region. In some embodiments, the nuclease lacks DNA strand cleavage activity. In some embodiments, the first regulatory element is a polymerase III promoter. In some embodiments, the second regulatory element is a polymerase II promoter.

Any useful Cas enzyme or complex can be employed. Exemplary Cas enzymes or complexes include those involved in Class 1 or Class 2 CRISPR/Cas systems, as well as Type I, Type II, Type III, Type IV, Type V, or Type VI CRISPR/Cas systems, including but not limited to those requiring a single Cas protein during crRNA interference (e.g., such as in Class 1 systems); Cas9 (formerly known as Csn1 or Csx12, e.g., such as in Type II systems, including Type II-A and Type II-C systems); Cas12 (e.g., such as in Type V systems); Cas13 (e.g., such as in Type VI systems); the CRISPR-associated complex for antiviral defense (Cascade, including a RAMP protein); those requiring multiple Cas proteins during crRNA interference (e.g., such as in Class 2 systems); Cas3, Cas 7, Cas6, Cas5, Cas11, and/or Cas8 (e.g., for Type I systems, such as Type I-E systems); Csm (e.g., in Type III-A systems); Cmr (e.g., in Type III-B systems); Cas7, Cas5, Cas11, Cas10, and/or Cas6 (e.g., in Type III systems); Cas7, Cas5, Cas11, Cas8, DinG (or CysH), and/or Cas6 (e.g., in Type IV systems), as well as subassemblies or sub-components thereof and assemblies including such Cas enzymes or complexes. Additional Cas enzymes and complexes are described in Makarova K S et al., “Evolution and classification of the CRISPR—Cas systems,” Nat. Rev. Microbiol. 2011; 9:467-77; and Wang J Y et al., “Structural biology of CRISPR-Cas immunity and genome editing enzymes,” Nat. Rev. Microbiol. 2022; doi.org/10.1038/s41579-022-00739-4 (16 pp.), each of which is incorporated herein by reference in its entirety.

In some embodiments, the Cas enzyme is a variant, as compared to a corresponding wild-type enzyme, that lacks the ability to cleave both strands of double-stranded structure. For example, an aspartate-to-alanine substitution (D10A) in the RuvC I catalytic domain of Cas9 from S. pyogenes converts Cas9 from a nuclease that cleaves both strands to a nickase that cleaves a single strand. Other examples of mutations that render Cas9 a nickase include, without limitation, H840A, N854A, and/or N863A.

Further cleavage enzymes and cleavage conditions can include any useful characteristic, such as those described in U.S. Pat. No. 5,316,908 or 8,273,863; U.S. Pat. Pub. Nos. US 2019/0203242, US 2007/0178482, or US 2019/0002868; Int. Pub. No. WO 2020/232286; Bush et al., Molecules 2020 Jul. 26; 25(15):3386; Gu et al., Biotechniques 2013 June; 54(6):337-43; Cooney, Mol. Biotechnol. 1994; 2(2):119-127; Parker et al., Proc. Nat'l Acad. Sci. USA 1977; 74(3):851-855; Polyarush et al., Chem. Natural Compounds 2003; 39(6):592-594; Lan et al., J. Nucleic Acids 2012; 2012: 254630; Wang et al., J. Nucleic Acids 2010; 2010:421803 (3 p.); Amills et al., Genet Anal. 1996; 13:147-9; Abdel-Fattah et al., Biotechnology. 2006; 5:166-172; Chang et al., J. Biochem. Biophys. Methods 2008; 70:1199-202; Wang et al., J. Nucleic Acids 2010; 2010:421803; Wu et al., Mol. Biol. Rep. 2011; 38:2729-31; Swartjes et al., Biochem. Soc. Trans. 2020 Feb. 28; 48(1):207-219; Ma et al., Mol Cell. 2015 Nov. 5; 60(3):398-407; Hunt et al., Front. Mol. Biosci. 29 Apr. 2021 (accessible at doi.org/10.3389/fmolb.2021.670940); Makarova et al., Nat. Rev. Microbiol. 2011; 9:467-77; Wang et al., Nat. Rev. Microbiol. 2022; doi.org/10.1038/s41579-022-00739-4 (16 pp.); and/or Thermo Fisher Scientific, “Production of Single-stranded Circular DNA Molecules from Supercoiled Double-stranded Plasmids in vitro,” 2012, 1 page (accessible at tools.thermofisher.com/content/sfs/brochures/76-circular-ssdna-from-supercoiled-double-stranded-plasmids-in-vitro.pdf), each of which is incorporated herein by reference in its entirety.

### Kits

The present disclosure also provides kits for a single-stranded nucleic acid ladder (e.g., an ssDNA ladder). Such kits can include single-stranded nucleic acid (e.g., ssDNA) size standards, which include one or multiple standards with specific sizes.

The kit can also include instructions for practicing any of the methods described herein. Instructions supplied in the kits of the present disclosure are typically written instructions on a label or a package insert. The kits provided herein are in suitable packaging. Suitable packaging includes, but is not limited to, containers, bottles, vials, and flexible packaging. Kits can include additional components such as interpretive information.

### EXAMPLES

### Example 1: ssDNA Ladder Preparation Based on DNA Oligonucleotide-Guided Cleavage by a Restriction Enzyme

The microfluidic chip system from LabChip® GX Touch™ instrument (Part #CLS137031, PerkinElmer, Inc., Waltham, MA) was used in this example. Oligonucleotides used herein as guided oligonucleotides (guides) were synthesized by Integrated DNA Technologies, Inc. (Coralville, IA). Sequences for guides are provided in Table 1, in which restriction sequences are highlighted in bold. M13mp18 single-stranded DNA, restriction enzymes, and buffers were purchased from New England Biolabs (Ipswich, MA).

TABLE 1
Sequences of guided oligonucleotide

| Name | Sequences (5′-3′) |
|---|---|
| LM-HIND3 | AGT GCC AAG CTT GCA TGC CTG CAG (SEQ ID NO: 2) |
| LM-PAC1 | CGT CGC TAT TAA TTA ATT TTC CCT (SEQ ID NO: 3) |
| LM-AFEI | GGG TAA TTG AGC GCT AAT ATC AGA (SEQ ID NO: 4) |

One μg of M13mp18 DNA and 1 μM of short, guided oligonucleotides in 1×NEB rCutSmart™ buffer were firstly annealed at 90° C. for 4 minutes (min), then at 70° C. for 10 min, and finally ramped down to 37° C. at 0.1° C./sec for 20 min in a 50 μL volume. Twenty units (20U) of HindIII and 12.5 U of PacI or AfeI were then added into these annealed products and incubated at 37° C. for 90 min for cleavage according to Table 2 for different experimental conditions. After cleavage, the reaction product was incubated at 95° C. for 5 min for inactivation. Restriction enzyme-digested products were further purified with 2× AMPure XP beads protocol (cat #A63880, Beckman Coulter Inc., Brea, CA). Purified samples were mixed with equal volumes of 1× Sample buffer (from LabChip® RNA Pico reagent, part #CLS960012, PerkinElmer), heated at 70° C. for 2 min, then immediately chilled on ice for another 5 min before analysis using LabChip® RNA Pico reagent on LabChip® GX Touch™ instrument.

TABLE 2
Experimental conditions and expected size

| Condition | HindIII | PacI | AfeI | Expected size |
|---|---|---|---|---|
| Enzyme (−) | | | | Intact circular ssDNA |
| HindIII + PacI | X* | X | | Linear ssDNA, 2.1 kb (1^{#}), |
| | | | | 5.1 kb (2) |
| HindIII + AfeI | X | | X | Linear ssDNA, 3.2 kb (3), |
| | | | | 4.0 kb (4) |
| HindIII + AfeI + | X | X | X | Linear ssDNA, 1.1 kb (5), |
| PacI | | | | 2.1 kb (1), 4.0 kb (2) |

*Add enzyme for cleavage;
^{#}size labeling in FIG. 2

The results are shown in FIG. 2. In particular, FIG. 2A shows the location of restriction sites for the following restriction enzymes: HindIII at 6281, PacI at 4135, and AfeI at 3040. The expected cleavage pattern with different combination of restriction enzymes is shown in Table 2.

FIG. 2B shows the electropherogram results, and FIG. 2C shows the corresponding gel image output from LabChip® Reviewer software. The following observations are provided:
Condition Enzyme (−): without enzyme cleavage intact circular ssDNA is slightly visible at around 70 seconds (sec). This is the negative control of this experiment settings.
Condition HindIII+PacI cleavage: size 1 (2.1 kb) and size 2 (5.1 kb) are clearly visible at around 50 sec and 66 sec, respectively.
Condition HindIII+AfeI cleavage: size 3 (3.2 kb) and size 4 (4.0 kb) are clearly visible at around 58 sec and 62 sec, respectively.
Condition HindIII+PacI+AfeI cleavage: size 5 (1.1 kb), size 1 (2.1 kb), and size 4 (4.0 kb) are clearly visible at around 44 sec, 50 sec, and 62 sec, respectively.

In summary, the electrophoresis results meet the expectation of cleavage patterns based on the location of restriction enzymes. These results demonstrate non-limiting methods for preparation of ssDNA ladder compositions based on oligonucleotide-guided cleavage by restriction enzymes.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. All references (including those listed above), scientific articles, patent publications, and any other documents cited herein are hereby incorporated by reference for the substance of their disclosure.

1. A method for preparing a ladder composition, the method comprising:
providing a template comprising a single-stranded nucleic acid; and
incubating the template in the presence of a guided oligonucleotide and a cleavage enzyme,
wherein the guided oligonucleotide is configured to bind to a target region of the template, and wherein the cleavage enzyme is configured to bind to the guided oligonucleotide and to cleave a location within the target region, thereby generating the ladder composition comprising a plurality of cleaved, single-stranded nucleic acids.

2. The method of claim 1, wherein the template comprises a single-stranded deoxyribonucleic acid (ssDNA), and wherein the ladder composition comprises a plurality of cleaved ssDNA.

3. The method of claim 1, wherein the template comprises a natural construct, a synthetic construct, a linear construct, and/or a circular construct.

4. The method of claim 1, wherein the template comprises more than 500, 1000, 2000, or more bases.

5. The method of claim 1, wherein the target region comprises a restriction sequence, a recognition sequence, a protospacer region, or a reverse complement thereof.

6. The method of claim 5, wherein the location within the target region to be cleaved by the cleavage enzyme comprises a location within the restriction sequence, the recognition sequence, the protospacer region, or the reverse complement thereof.

7. The method of claim 1, wherein the guided oligonucleotide comprises a single-stranded deoxyribonucleic acid (ssDNA), a single-stranded ribonucleic acid (ssRNA), or a single-stranded DNA/RNA hybrid (ssDNA/RNA).

8. The method of claim 1, wherein the guided oligonucleotide comprises a restriction sequence, a recognition sequence, a protospacer region, or a reverse complement thereof.

9. The method of claim 1, wherein the template comprises a protospacer adjacent motif (PAM) sequence, or a reverse complement thereof, in proximity to the target region; and/or wherein the guided oligonucleotide comprises a PAM sequence or a reverse complement thereof.

10. The method of claim 9, wherein the guided oligonucleotide further comprises a CRISPR ribonucleic acid (crRNA) region and/or a trans-acting CRISPR RNA (tracrRNA) region region; and optionally wherein the guided oligonucleotide further comprises a linker disposed between the crRNA and tracrRNA regions

11. The method of claim 1, wherein the cleavage enzyme comprises a restriction enzyme, a nicking enzyme, a programmable endonuclease, an Argonaute protein, a CRISPR-associated (Cas) enzyme, a nuclease, or a variant thereof.

12. The method of claim 1, wherein said providing comprises, prior to said incubating:
providing the guided oligonucleotide to the template, thereby providing a bound template, and
further providing the cleavage enzyme to the bound template.

13. The method of claim 1, wherein said providing comprises, prior to said incubating:
providing a pre-assembled complex to the template, wherein the pre-assembled complex comprises the guided oligonucleotide bound to the cleavage enzyme.

14. The method of claim 1, wherein said providing comprises, prior to said incubating:
providing the guided oligonucleotide and the cleavage enzyme to the template, wherein the guided oligonucleotide and the cleavage enzyme can be provided in any order or at the same time.

15. The method of claim 1, wherein at least 80%, 90%, or more of a population comprising the plurality of cleaved, single-stranded nucleic acids in the ladder composition is single-stranded.

16. The method of claim 1, wherein at least 50%, 60%, 70%, 80%, or more of a population comprising the plurality of cleaved, single-stranded nucleic acids has a length of 500, 600, 700, 800, 900, 1000, 2000, or more bases.

17. The method of claim 1, wherein said incubating is conducted in the absence of albumin or a blocking agent.

18. The method of claim 1, wherein the ladder composition is not further purified nor diluted.

19. A ladder composition comprising a population comprising a plurality of cleaved, single-stranded nucleic acids,
wherein at least 80%, 90%, or more of the population is single-stranded; and/or
wherein at least 50%, 60%, 70%, 80%, 90%, or more of the population has a length of 500, 600, 700, 800, 900, 1000, 2000, or more bases.

20. The ladder composition of claim 19, wherein the population comprises a plurality of cleaved, single-stranded deoxyribonucleic acids (ssDNAs).

21. The ladder composition of claim 19, wherein the composition is prepared by the method of claim 1.

22. A kit comprising:
a first ladder composition comprising the ladder composition of claim 19; and
instructions for using the first ladder composition in an assay.

23. The kit of claim 22, further comprising:
a second ladder composition comprising the ladder composition of claim 19,
wherein the first and second ladder compositions are different, and wherein the instructions further comprise instructions for using the second ladder composition in the assay.

24. The kit of claim 23, wherein the first ladder composition comprises a first population comprising a plurality of first cleaved, single-stranded nucleic acids; wherein the second ladder composition comprises a second population comprising a plurality of second cleaved, single-stranded nucleic acids; and wherein at least 90% of the first population has a length that is different from at least 90% of the second population.

25. The kit of claim 22, wherein the assay comprises electrophoresis.


SEQUENCE LISTING (4 sequences)

SEQ ID NO: 1 - DNA, 17 bp, synthetic construct
  source 1..17: mol_type = other DNA
ttaccgctaatggtgtg

SEQ ID NO: 2 - DNA, 24 bp, synthetic construct
  source 1..24: mol_type = other DNA
agtgccaagcttgcatgcctgcag

SEQ ID NO: 3 - DNA, 24 bp, synthetic construct
  source 1..24: mol_type = other DNA
cgtcgctattaattaattttccct

SEQ ID NO: 4 - DNA, 24 bp, synthetic construct
  source 1..24: mol_type = other DNA
gggtaattgagcgctaatatcaga